(19) United States
(12) Patent Application Publication
(10) Pub. No.: US 2025/0266134 A1
(43) Pub. Date: Aug. 21, 2025

(54) APPARATUS AND METHOD FOR PREDICTING QUALITY OF SLURRY

(71) Applicant: ELECTRONICS AND TELECOMMUNICATIONS RESEARCH INSTITUTE, Daejeon (KR)

(72) Inventors: Hyun Woo OH, Daejeon (KR); Hyun Jong KIM, Daejeon (KR); Seung Woog JUNG, Daejeon (KR); Kang Il CHOI, Daejeon (KR); Yo Han CHOI, Daejeon (KR)

(21) Appl. No.: 19/006,492

(22) Filed: Dec. 31, 2024

(30) Foreign Application Priority Data
Feb. 20, 2024 (KR) 10-2024-0024514

(51) Int. Cl. G16C 60/00; G01N 29/02; G01N 29/44; G01N 33/38

(52) U.S. Cl. CPC G16C 60/00; G01N 29/02; G01N 29/4418; G01N 29/4472; G01N 33/388; G01N 2291/02416

(57) ABSTRACT

Provided is an apparatus and method for predicting quality of slurry. The apparatus includes: a data collection module collecting at least one of data among raw and secondary material data, process condition data, environmental data, and measurement data in a ball mill process; a contact temperature sensor attached to outside of a ball mill facility to measure a facility temperature; a vibration sensor detecting vibration from the outside of the ball mill facility; a database; and a processor coupled to the data collection module, the contact temperature sensor, the vibration sensor, and the database, wherein the processor stores the data collected through the data collection module with a contact temperature measured by the contact temperature sensor and vibration data detected by the vibration sensor, generates and trains a learning model, and predicts quality of slurry based on the contact temperature and the vibration data in the ball mill process.

### CROSS-REFERENCE TO RELATED APPLICATION

This application claims priority to and the benefit of Korean Patent Application No. 10-2024-0024514, filed on Feb. 20, 2024, the disclosure of which is incorporated herein by reference in its entirety.

### BACKGROUND

### 1. Field of the Invention

The present invention relates to an apparatus and method for predicting the quality of slurry, and more specifically, to an apparatus and method for predicting the quality of slurry that are capable of predicting the quality of slurry in a ceramic ball mill process.

### 2. Description of Related Art

In general, among ceramic materials, advanced ceramic materials are increasingly being utilized across fields such as electronics, mechanical structures, energy and the environment, and biotechnology applications. Specific processes for producing products for advanced ceramics may vary depending on their applications, but representative processes include ball milling, spray drying, molding, and sintering.

A characteristic of the ceramic industry is that ceramic powder may be produced through ball milling and spray drying, and the ceramic powder may form a semi-finished product or a product in itself. Ceramic powder may be purchased and only molded for shipment, or the entire process from the ball milling process to the sintering process may be performed to produce products.

The quality of a semi-finished product or product produced in each unit process varies depending on the raw materials and process conditions applied in each unit process. The quality of the semi-finished product produced in one unit process may be an input variable in the next process. In other words, the quality of the slurry produced in the ball milling process significantly affects the production quality of the spray drying process.

The ball mill process is a process in which ceramic particle raw materials are put into a ball mill container together with water, additives, ceramic balls, and the like, and are rotated for the ceramic particles to be crushed into small pieces. While being rotated in the ball mill container, the ceramic balls collide with each other to crush the ceramic particles into small pieces.

The ceramic raw materials used in the ball mill process vary depending on the product, and the used additives, such as dispersants, binders, plasticizers, release agents, and wetting agents, also vary.

In this case, when a large amount of dispersants is added, the ceramic slurry is loosened, but the slurry viscosity decreases, which may affect the particle size of the slurry. In addition, when a large amount of binders is added, the slurry viscosity increases, potentially reducing the collision of ceramic balls, which may result in less crushing of ceramic particles. In addition, when a large amount of solvent (water) is added, the slurry viscosity decreases, and the size and number of ceramic balls also affect the slurry viscosity and particle size.

Meanwhile, the temperature of the slurry may be determined by the frequency of collisions between the ceramic balls to form the slurry. When the temperature of the slurry is too high, the dispersant may not work, which causes the slurry to clump like dough and rotate inside the ball mill.

Such many variables determine the quality of the slurry, but there is no method of identifying what phenomenon occurs inside the ball mill, how the viscosity, particle size, and temperature of the slurry are changing, and whether the slurry is produced according to a quality level.

In the related art titled, “Real-time collection and correlation analysis of viscosity and acoustic data during ball milling process” in J.Korean Powder Metall. Inst., vol.27, No.6, 484-489, 2020, data related to acoustics, temperature, power consumption, and rotation speed is collected during the ball milling process of dispersing alumina in a solvent, and changes in the characteristics of the alumina-dispersed slurry, such as the slurry viscosity and particle size, are measured in real time.

By comparing the above collected data with the changes in the characteristics of the slurry, the correlation is derived, and it is confirmed that when the ball mill device is configured in a circulating manner, the viscosity of the internal slurry increases and the particle size decreases with the milling time, and that the acoustic data changes with the milling time.

In the related art, it is confirmed only that the slurry viscosity and particle size change with the milling time, and that the acoustic data changes with the milling time. However, the quality prediction of the slurry viscosity, slurry particle size, and slurry temperature, which are important in the field, has not been addressed.

In addition, the related art adopts a method in which a hole is drilled in a rotating shaft of a ball mill facility and a slurry inside a ball mill container is extracted using a circulation pump to measure the slurry viscosity, slurry particle size, and slurry temperature, and then re-injected into the ball mill container. The method is not preferred due to the burden of modifying the ball mill device in the field, and a measuring device for measuring the viscosity and particle size of the slurry is expensive, and thus has a difficulty in introduction to small and medium-sized enterprises.

In addition, there have been cases in which vibration sensors or acoustic sensors are attached and used, but it is difficult to introduce acoustic sensors due to the environment in which noise is generated from many facilities operating at the factory site, and with regard to vibration sensors, there have been attempts to observe correlation and connection, but it is not possible to conduct experiments for all combinations of many variables, such as raw materials, solvents, additives, ceramic balls, and process conditions, and there has been no published case in which the quality of slurry is predicted through such experiments, which makes it difficult to apply the method.

The background art of the present invention is disclosed in Korean Registered Patent No. 10-2336651 (Dec. 2, 2021).

### SUMMARY OF THE INVENTION

The present invention is directed to providing an apparatus for predicting the quality of slurry in which a training dataset is configured by linking vibration data measured on the outside of a ball mill facility, temperature data measured in an adhesive manner on the outside of the ball mill facility, and the temperature of slurry measured by inserting a thermometer into a lid of the ball mill facility, and then the viscosity, particle size, and temperature of slurry in a ceramic ball mill process are predicted based on adhesive temperature data and vibration data using artificial intelligence (AI), and a method thereof.

According to an aspect of the present invention, there is provided an apparatus for predicting quality of slurry, which includes: a data collection module that collects at least one type of data among raw and secondary material data, process condition data, environmental data, and measurement data in a ball mill process; a contact temperature sensor attached to an outside of a ball mill facility to measure a facility temperature; a vibration sensor that detects vibration from the outside of the ball mill facility; a database; and a processor operatively coupled to the data collection module, the contact temperature sensor, the vibration sensor, and the database, wherein the processor stores the data collected through the data collection module together with a contact temperature measured by the contact temperature sensor and vibration data detected by the vibration sensor in the database, then generates and trains a learning model, and then predicts quality of slurry based on the contact temperature and the vibration data in the ball mill process.

The data collection module may include a slurry measuring device that extracts slurry from the ball mill facility to measure a viscometer slurry viscosity, a granulometer slurry particle size, and a viscometer slurry temperature, and a slurry temperature measuring device that forms a measuring hole in a lid of the ball mill facility and measures a lid hole slurry temperature with a temperature sensor inserted into the measuring hole.

The measuring hole may have a size allowing the slurry to pass therethrough while preventing a ceramic ball from passing therethrough.

The processor may calculate a correction value between the viscometer slurry temperature measured by the slurry measuring device and the lid hole slurry temperature measured by the slurry temperature measuring device, generate a mathematical model through correlation analysis linking the lid hole slurry temperature measured by the slurry temperature measuring device with the contact temperature measured by the contact temperature sensor in a test process, and then predict a slurry temperature based on the contact temperature of the contact temperature sensor in the ball mill process.

The processor may generate a mathematical model for the lid hole slurry temperature according to a ball mill time, generate a mathematical model for the viscometer slurry temperature according to the ball mill time, calculate an external influence degree through the mathematical model for the lid hole slurry temperature to correct the mathematical model for the viscometer slurry temperature, and calculate a temperature rise of the slurry from the corrected mathematical model for the viscometer slurry temperature to supplement the mathematical model with the lid hole slurry temperature.

The quality of the slurry may include at least one of a slurry viscosity, a slurry particle size, and a slurry temperature.

According to an aspect of the present invention, there is provided a method of predicting quality of slurry, which includes: collecting, by a processor, at least one type of data among raw and secondary material data, process condition data, environmental data, and measurement data through a data collection module in a ball mill process; storing, by the processor, the collected data in the database; generating and training, by the processor, a learning model based on the data stored in the database; receiving, by the processor, a contact temperature and vibration data; and predicting, by the processor, quality of slurry based on the contact temperature and the vibration data through the learning model.

The predicting of the quality of the slurry may include calculating, by the processor, a correction value between a viscometer slurry temperature measured by a slurry measuring device and a lid hole slurry temperature measured by a slurry temperature measuring device, generating a mathematical model through correlation analysis linking the lid hole slurry temperature measured by the slurry temperature measuring device with the contact temperature measured by a contact temperature sensor in a test process, and then predicting a slurry temperature based on the contact temperature of the contact temperature sensor in a ball mill process.

The predicting of the quality of the slurry may include generating, by the processor, a mathematical model for the lid hole slurry temperature according to a ball mill time, generating a mathematical model for the viscometer slurry temperature according to the ball mill time, calculating an external influence degree through the mathematical model for the lid hole slurry temperature to correct the mathematical model for the viscometer slurry temperature, and calculating a temperature rise of the slurry from the corrected mathematical model for the viscometer slurry temperature to supplement the mathematical model with the lid hole slurry temperature.

The quality of the slurry may include at least one of a slurry viscosity, a slurry particle size, and a slurry temperature.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram showing an apparatus for predicting the quality of slurry according to one embodiment of the present invention;

FIG. 2 is an exemplary diagram showing a measuring device for measuring a slurry temperature in an apparatus for predicting the quality of slurry according to one embodiment of the present invention;

FIGS. 3A to 3C are graphs showing a slurry temperature measured in an apparatus for predicting the quality of slurry according to one embodiment of the present invention; and

FIG. 4 is a flowchart for describing a method of predicting the quality of slurry according to one embodiment of the present invention.

### DETAILED DESCRIPTION OF EXEMPLARY EMBODIMENTS

Hereinafter, an apparatus and method for predicting the quality of slurry according to the present invention will be described in detail with reference to the accompanying drawings. The thickness of each line or the size of each component shown in the drawings may be exaggerated for the purposes of clarity and convenience. Although terms used herein are selected from among general terms that are currently widely used in consideration of functions in the exemplary embodiments, these may be changed according to intentions or customs of those skilled in the art or the advent of new technology.

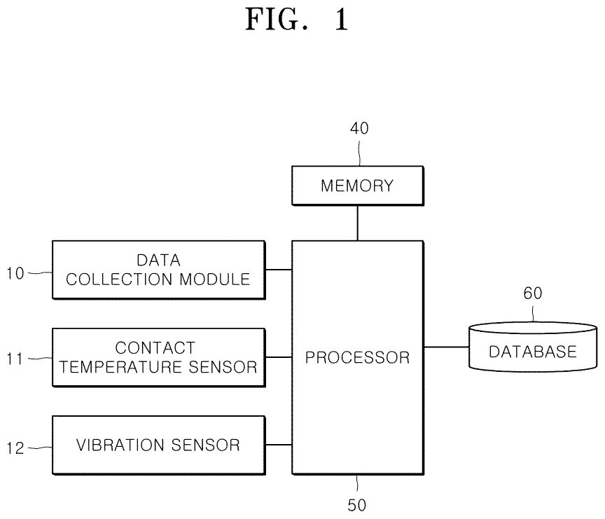

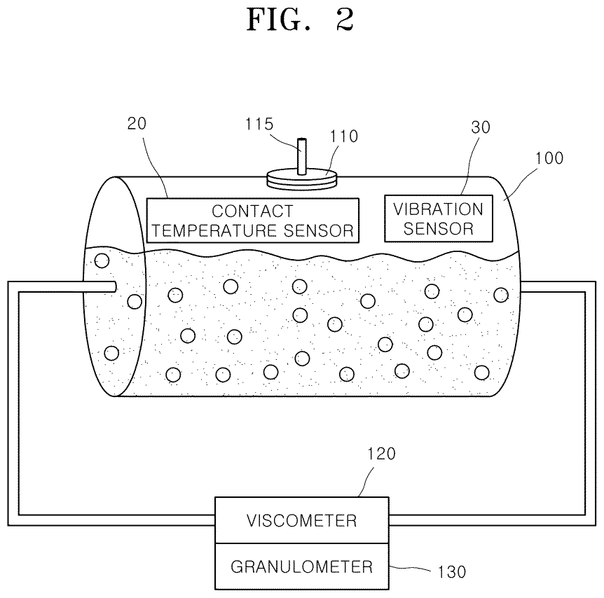

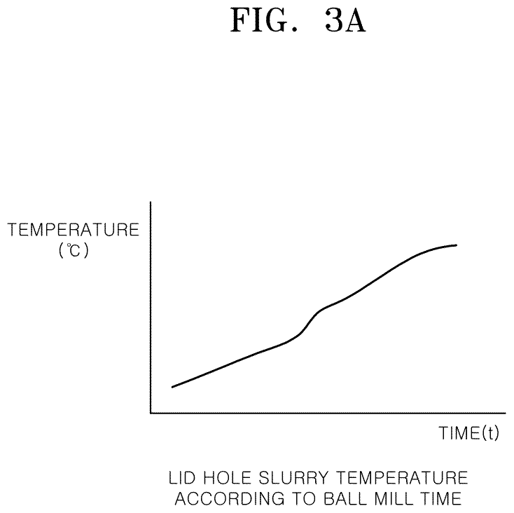

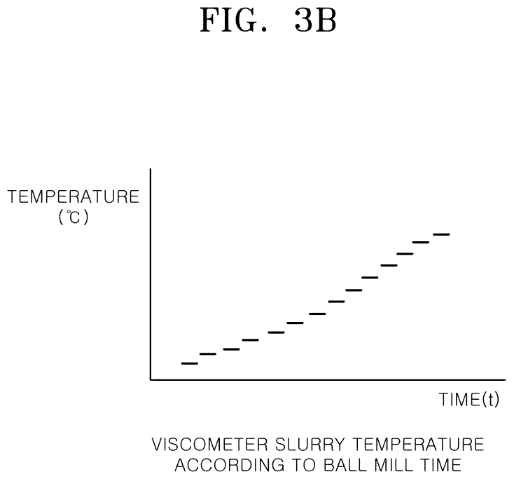

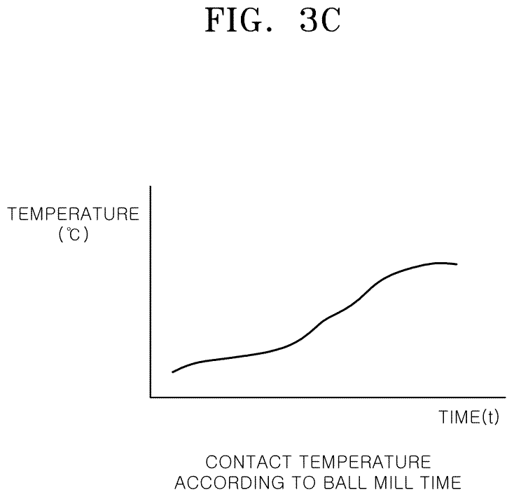

FIG. 1 is a block diagram showing an apparatus for predicting the quality of slurry according to one embodiment of the present invention, FIG. 2 is an exemplary diagram showing a measuring device for measuring a slurry temperature in an apparatus for predicting the quality of slurry according to one embodiment of the present invention, and FIG. 3 is a graph showing a slurry temperature measured in an apparatus for predicting the quality of slurry according to one embodiment of the present invention.

Referring to FIGS. 1 to 3, the apparatus for predicting the quality of slurry according to the embodiment of the present invention may include a data collection module 10, a contact temperature sensor 20, a vibration sensor 30, a database 60, a memory 40, and a processor 50.

The data collection module 10 may collect at least one type of data among raw and secondary material data, process condition data, environmental data, and measurement data in a ball mill process to construct factors that may affect the quality of slurry of the ball mill process as a dataset for training.

The raw and secondary material data may include the capacity of a ball mill, the input quantity of a ceramic particle raw material put into the ball mill container, the manufacturer of the ceramic particle raw material, impurities included in the ceramic particle raw material, the purity of the raw material, and other characteristics of the raw material.

In this case, water or an organic solvent is used as a solvent, and the characteristics of the solvent may include the manufacturer, purity, impurity types, and the like.

In addition, dispersants, binders, plasticizers, release agents, and wetting agents are additives, and since the characteristics of the additives may vary depending on the ratios at which the additives are added, the manufacturer, purity, and impurity type may be included as the input quantities and the characteristics of the additives.

In addition to the additives, the temperature of water used as a solvent may affect the quality of slurry. In the field, during winter, the temperature of water may be an important factor as cold water is not used and hot water is mixed in.

The ball mill process includes putting raw materials into a solvent together with additives and adding ceramic balls to finely crush the ceramic particles. In this case, depending on the type, size, and amount of the ceramic balls added, the degree to which the ceramic particles are crushed as the ceramic balls collide during rotation varies.

Therefore, even when the same raw and secondary materials and the same ceramic balls are added, process condition data, such as an actual rotation speed (RPM) of the ball mill, a time to rotate the ball mill container at the RPM, and an elapsed time since the ball mill process started may be included.

In addition, at the manufacturing site, even when the process is performed under the same process conditions with the same raw and secondary materials, slurry of the same quality may not be produced.

Among such cases, representative factors affecting the quality of the slurry may include environmental temperature and humidity.

In particular, since the ball mill process is performed inside the ball mill container, it is difficult to check the state in which the slurry is being produced inside. Accordingly, referring to FIG. 2, while holes are formed by drilling both sides of a rotation shaft of a ball mill container 100, a slurry measuring device configured to extract a slurry with a pump to measure a slurry viscosity and a particle size of a current state through a viscometer 120 and a granulometer 130, and then re-inject the slurry through the opposite side may be used to collect a viscometer slurry viscosity, a granulometer slurry particle size, and a viscometer slurry temperature.

In addition, referring to FIG. 2, a slurry temperature measuring device that forms a measuring hole in a lid 110 of the ball mill facility and measures a lid hole slurry temperature using a temperature sensor 115 inserted into the measuring hole may be configured to measure the temperature of a slurry.

In this case, the measuring hole is formed to a size that allows the slurry to pass therethrough while preventing the ceramic balls from passing therethrough, so that when the ball mill container rotates and the slurry is introduced through the measuring hole, the temperature of the slurry is measured through the temperature sensor 115, and then the measured lid hole slurry temperature may be wirelessly output.

The vibration data measured through the vibration sensor 30 on the outside of the ball mill facility may be affected by the capacity of a ball mill, the amount of raw materials and the input quantity of a solvent, the degree of dispersion of the slurry by the dispersant, the size and input quantity of ceramic balls, and the like, and may be affected by the RPM, which is the rotation speed of the ball mill. Therefore, pattern analysis of vibration data may be correlated with the data measured by a continuous viscometer.

As described above, the data collection module 10 may collect, as raw and secondary material data, at least one of a capacity of a ball mill, an input quantity of raw materials, characteristics of the raw materials, an input quantity of a solvent, characteristics of the solvent, an input quantity of a dispersant, characteristics of the dispersant, characteristics of the binder, an input quantity of a plasticizer, characteristics of the plasticizer, an input quantity of a release agent, characteristics of the release agent, an input quantity of a wetting agent, and characteristics of the wetting agent.

In addition, the data collection module 10 may collect, as process condition data, at least one of the type of ceramic balls, the size of ceramic balls, the number of ceramic balls, the RPM of the facility, the ball mill time, and the ball mill start time, and as environmental data, at least one of an environmental temperature and an environmental humidity.

In addition, the data collection module 10 may collect, as measurement data, at least one of vibration data, a lid hole slurry temperature, a contact temperature, a viscometer slurry temperature, a viscometer slurry viscosity, and a granulometer slurry particle size.

The contact temperature sensor 20 may be attached to the outside of the ball mill container 100, which is a ball mill facility, as shown in FIG. 2, and may measure a facility temperature and provide the measured facility temperature.

The vibration sensor 30 may detect vibration from the outside of the ball mill container 100, which is a ball mill facility, as shown in FIG. 2, and provide the detected vibration.

The database 60 may store not only the data collected through the data collection module 10, but also a contact temperature measured by the contact temperature sensor 20 and vibration data detected by the vibration sensor 30, and may also store training data and the results of training.

The memory 40 may store an execution program related to the operation of the apparatus for predicting the quality of slurry, and the stored information may be selected by the processor 50 as needed.

That is, various types of data and commands generated during the execution of an operating system (O/S) or an application (a program or applet) for driving the apparatus for predicting the quality of slurry are stored in the memory 40. In this case, the memory 40 may be implemented as a nonvolatile memory, a volatile memory, a flash memory, a hard disk drive (HDD), or a solid state drive (SSD). In addition, the memory 40 may be accessed, and data may be read/recorded/modified/deleted/updated by the processor 50.

The processor 50 is operatively coupled to the data collection module 10, the contact temperature sensor 20, the vibration sensor 30, the database 60, and the memory 40, and may copy various programs, which are stored in the memory 40 to control the overall operation of the apparatus for predicting the quality of slurry, to a random access memory (RAM) and execute the programs to perform various operations.

In addition, the processor 50 may have configurations for performing each function that are divided at hardware, software, or logic levels. In this case, dedicated hardware for performing each function may be used. To this end, the processor 50 may be implemented as at least one of an application specific integrated circuit (ASIC), a digital signal processor (DSP), a programmable logic device (PLD), a field programmable gate array (FPGA), a central processing unit (CPU), a microcontroller, and/or a microprocessor, or may include at least one of the same.

The processor 50 may be implemented as a CPU or a system on chip (SoC), may control a plurality of hardware or software components connected to the processor 50 by driving an operating system or an application and may perform various data processing and operations. The processor 50 may be configured to execute at least one command stored in the memory 40 and store the execution result data in the memory 40.

The processor 50 stores data collected through the data collection module 10 together with the contact temperature measured by the contact temperature sensor 20 and the vibration data detected by the vibration sensor 30 in the database 60, then generates and trains a learning model, and then predicts the quality of slurry based on the contact temperature and the vibration data in the ball mill process.

In this case, the processor 50 calculates a correction value between a viscometer slurry temperature measured by the slurry measuring device and a lid hole slurry temperature measured by the slurry temperature measuring device among data collected through the data collection module 10, generates a mathematical model through correlation analysis linking the lid hole slurry temperature measured by the slurry temperature measuring device and the contact temperature measured by the contact temperature sensor in a test process, and then predicts a slurry temperature based on the contact temperature of the contact temperature sensor 20 in a ball mill process.

More specifically, the processor 50 constructs a mathematical model by deriving a formula for the lid hole slurry temperature according to the ball mill time as shown in FIG. 3A, constructs a mathematical model by deriving a formula for the viscometer slurry temperature according to the ball mill time as shown in FIG. 3B, and then constructs a mathematical model obtained through correlation analysis linking the contact temperature according to the ball mill time by using the correlation between the mathematical models as a correction value as shown in FIG. 3C, thereby predicting the slurry temperature based on the contact temperature.

That is, the viscometer slurry temperature according to the ball mill time is measured as shown in FIG. 2 in which holes are formed in the ball mill facility to extract the slurry with a pump and measure the viscometer slurry temperature through the viscometer 120, and thus experimental data may be collected through a test process that builds a testbed.

In this case, the viscometer slurry temperature measured by the viscometer 120 according to the ball mill time is collected as values measured at predetermined time intervals (1 minute, 5 minutes, 10 minutes, etc.). Therefore, the viscometer slurry temperature may be expressed as a single value for each measurement period as shown in FIG. 3B. Therefore, the corresponding values may be connected as a straight line or a curve, and a formula representing the connected values may be derived.

For example, the processor 50 may derive a formula through Excel, or may derive a formula using a program such as Sigmaplot to construct a mathematical model.

However, the viscometer slurry temperature measured by the viscometer 120 according to the ball mill time is affected by an external factor while the slurry extracted from the ball mill reaches the viscometer 120 through a pipe, and thus the temperature of the slurry is subject to change.

Meanwhile, the lid hole slurry temperature collected from the slurry temperature measuring device may be detected in milliseconds through the temperature sensor 115 inserted into the measuring hole of the lid and may be expressed as a line over time as shown in FIG. 3A, so that a formula representing the corresponding values may be derived.

For example, the processor 50 may derive a formula through Excel, or may derive a formula using a program such as Sigmaplot to construct a mathematical model.

However, a rise in the lid hole slurry temperature may deviate from that of the actual slurry temperature. This is because when the ball mill container 100 rotates, the slurry may pass through the measuring hole and fill the measuring hole, and before the slurry is completely removed, the hole is refilled and emptied.

Therefore, by complementing the advantages and disadvantages of the viscometer slurry temperature and the lid hole slurry temperature according to the ball mill time, the temperature of the slurry inside the ball mill may be calculated.

The processor 50 may calculate the standard deviation between the data generated from the mathematical model of the lid hole slurry temperature according to the ball mill time and the data generated from the mathematical model of the viscometer slurry temperature and reflect the standard deviation as a correction value of the viscometer slurry temperature.

A rise of the slurry temperature obtained by calculating the slope at each point based on data regenerated from the mathematical model of the viscometer slurry temperature in which the external influence is reflected does not show a large deviation even when there is an external influence, and thus may be applied to the temperature of the slurry inside the ball mill.

Therefore, the processor 50 may apply the rise in the slurry temperature calculated from the slope of the viscometer slurry temperature to the mathematical model of the lid hole slurry temperature, thereby predicting the temperature of the slurry inside the ball mill through the mathematical model of the lid hole slurry temperature.

The ball mill process takes about 12 to 20 hours and operates at a rotation speed of about 40 RPM, and the slurry temperature increases to 40 to 50 degrees, transferring heat to the outside of the ball mill.

Therefore, by linking a contact temperature measured by attaching a contact temperature sensor 20 to the outside of the ball mill to avoid external influence with a lid hole slurry temperature, the slurry temperature may be predicted based on the contact temperature.

That is, the contact temperature may be expressed as a line according to the ball mill time as shown in FIG. 3D, and a formula that may represent the corresponding values may be derived.

For example, the processor 50 may derive a formula through Excel, or may derive a formula using a program such as Sigmaplot to construct a mathematical model.

Here, the contact temperature is a value based on heat gradually transmitted from the temperature of the slurry inside the ball mill and thus may be measured relatively low, but the deviation may be calculated by comparing the contact temperature with data generated from the mathematical model of the lid hole slurry temperature in which the slurry temperature rise is reflected.

Therefore, the processor 50 may predict the temperature of the slurry inside the ball mill from the contact temperature by calculating the deviation between data generated from the mathematical model of the contact temperature according to the ball mill time and the mathematical model of the lid hole slurry temperature in which the slurry temperature rise is reflected.

In addition, the processor 50 may derive a formula through a graph according to the deviation between the lid hole slurry temperature and the contact temperature and predict a change in the temperature of the slurry inside the ball mill based on the contact temperature measured by the contact temperature sensor 20, thereby predicting the actual slurry temperature.

Therefore, the slurry temperature inside the ball mill may be predicted by the modification of the lid of the ball mill facility in the field and measurement of the lid hole slurry temperature, together with the contact temperature of the contact temperature sensor 20 attached to the outside of the ball mill facility.

In addition, the processor 50 may generate a learning model based on the data collected through the data collection module 10.

For example, in the present embodiment, an AI model for generating the learning model may perform preprocessing techniques, correlation analysis, and influence analysis by utilizing an AutoML model that may be easily applied in the field.

However, the training dataset may include at least one of a capacity of a ball mill, an input quantity of a raw material, characteristics of the raw material, an input quantity of a solvent, characteristics of the solvent, an input quantity of a dispersant, characteristics of the dispersant, an input quantity of a binder, characteristics of the binder, an input quantity of a plasticizer, characteristics of the plasticizer, an input quantity of a release agent, characteristics of the release agent, an input quantity of a wetting agent and characteristics of the wetting agent as raw and secondary material data collected through the data collection module 10, at least one of the type of ceramic balls, the size of the ceramic balls and the number of ceramic balls, an RPM of facility, a ball mill time and a ball mill start time as process condition data, at least one of an environmental temperature and an environmental humidity as environmental data, and at least one of vibration data, a lid hole slurry temperature, a contact temperature, a viscometer slurry temperature, a viscometer slurry viscosity, and a granulometer particle size as measurement data.

Therefore, the processor 50 may perform preprocessing (missing value removal, etc.) and correlation analysis (MIC, MEV, TIC, MAS, MCN, PCC, LIME, SHAP, etc.) based on the training data configured as described above, generate a learning model using AutoML (H20, Auto-skearn, Pycaret, TPOT, and the like) and train the learning model, and then predict the quality of a slurry, including a slurry viscosity, a slurry particle size, and a slurry temperature, based on a contact temperature and vibration data in a ball mill process.

As described above, the apparatus for predicting the quality of slurry according to an embodiment of the present invention may construct a training dataset by linking vibration data measured on the outside of a ball mill facility and temperature data measured in an adhesive manner on the outside of the ball mill facility with the temperature of a slurry measured by inserting thermometer into a lid of the ball mill facility, and then predict the viscosity, particle size, and temperature of the slurry in a ceramic ball mill process based on the adhesive temperature data and the vibration data using artificial intelligence (AI), thereby minimizing modification of the ball mill facility and enabling easy and convenient prediction of the quality of the slurry on site at low cost without requiring an expensive measuring device.

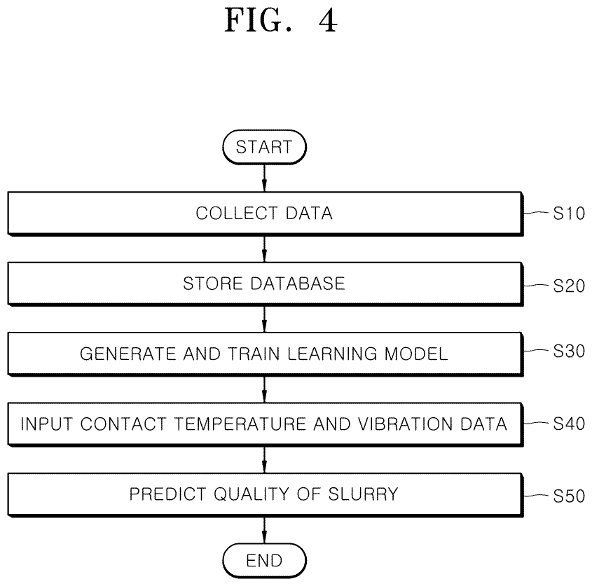

FIG. 4 is a flowchart for describing a method of predicting the quality of slurry according to one embodiment of the present invention.

Referring to FIG. 4, the method of predicting the quality of slurry according to the embodiment of the present invention includes executing, by the processor 50, an execution program stored in the memory 40 and collecting at least one type of data among raw and secondary material data, process condition data, environmental data, and measurement data in the ball mill process through the data collection module 10 (S10).

Here, the data collected through the data collection module 10 is data for configuring a training data set, and as raw and secondary material data, at least one of a capacity of a ball mill, an input quantity of raw materials, characteristics of the raw materials, an input quantity of a solvent, characteristics of the solvent, an input quantity of a dispersant, characteristics of the dispersant, characteristics of a binder, an input quantity of a plasticizer, characteristics of the plasticizer, an input quantity of a release agent, characteristics of the release agent, an input quantity of a wetting agent, and characteristics of the wetting agent may be collected.

In addition, as process condition data, at least one of the type of ceramic balls, the size of the ceramic balls, the number of ceramic balls, the RPM of a facility, the ball mill time, and the ball mill start time, and as environmental data, at least one of an environmental temperature and an environmental humidity may be collected.

In addition, as measurement data, at least one of vibration data, a lid hole slurry temperature, a contact temperature, a viscometer slurry temperature, a viscometer slurry viscosity, and a granulometer slurry particle size may be collected.

The processor 50, after collecting data to form the training data set in operation S10, stores the collected data in the database 60 (S20).

The processor 50, after storing the collected data in the database in operation S20, generates a learning model based on the data stored in the database 60 and trains the learning model to predict the quality of slurry based on a contact temperature and vibration data (S30).

For example, the processor 50 may perform preprocessing (missing value removal, etc.) and correlation analysis (MIC, MEV, TIC, MAS, MCN, PCC, LIME, SHAP, etc.) based on the configured training data, and generate a learning model using AutoML (H20, Auto-skearn, Pycaret, TPOT, etc.) and train the learning model.

The processor 50, after training the learning model to predict the quality of slurry in operation S30, receives a contact temperature and vibration data from the contact temperature sensor 20 and the vibration sensor 30 (S40).

Here, the contact temperature sensor 20 and the vibration sensor 30 may be attached to the outside of the ball mill container 100 of the ball mill facility as shown in FIG. 2 to measure the contact temperature and the vibration data.

The processor 50, after receiving the contact temperature and vibration data in operation S40, predicts a slurry temperature, a slurry viscosity, and a slurry particle size as the quality of the slurry through the learning model based on the input contact temperature and the input vibration data (S50).

Here, the slurry temperature may be predicted by the processor calculating a correction value between the viscometer slurry temperature measured by the slurry measuring device and the lid hole slurry temperature measured by the slurry temperature measuring device, generating a mathematical model through correlation analysis linking the lid hole slurry temperature measured by the slurry temperature measuring device and the contact temperature measured by the contact temperature sensor 20 in a test process, and then predicting a slurry temperature based on the contact temperature of the contact temperature sensor 20 in a ball mill process.

In this case, the processor 50 may generate a mathematical model for the lid hole slurry temperature according to a ball mill time, generate a mathematical model for the viscometer slurry temperature according to the ball mill time, calculate an external influence degree through the mathematical model for the lid hole slurry temperature to correct the mathematical model for the viscometer slurry temperature, and calculate a temperature rise of the slurry from the mathematical model for the viscometer slurry temperature to supplement the mathematical model with the lid hole slurry temperature.

As described above, the method of predicting the quality of slurry according to an embodiment of the present invention may configure a training dataset by linking vibration data measured on the outside of a ball mill facility and temperature data measured in an adhesive manner on the outside of the ball mill facility with the temperature of a slurry measured by inserting a thermometer into a lid of the ball mill facility, and then predict the viscosity, particle size, and temperature of the slurry in a ceramic ball mill process based on the adhesive temperature data and the vibration data using AI, thereby minimizing modification of the ball mill facility and enabling easy and convenient prediction of the quality of the slurry on site at a low cost without requiring an expensive measuring device.

As used herein, the term “module” may include units implemented in hardware, software, or firmware, and may be interchangeably used with terms such as “logic,” “logic block,” “component,” or “circuit.” The module may be an integrally configured component or a minimum unit or part of the integrally configured component that performs one or more functions. For example, according to one embodiment, the module may be implemented in the form of an application-specific integrated circuit (ASIC).

The implementations described herein may be implemented in, for example, a method or process, an apparatus, a software program, a data stream, or a signal. Even when only discussed in the context of a single form of implementation (for example, discussed only as a method), the implementation of discussed features may also be implemented in other forms (for example, an apparatus or program). An apparatus may also be implemented in appropriate hardware, software, and firmware. The methods may be implemented in, for example, an apparatus such as a processor, which is a general term for a processing device, such as a computer, a microprocessor, an integrated circuit, or a programmable logic device. Processors also include communication devices such as computers, cellular phones, portable/personal digital assistants (PDAs), and other devices that facilitate communication of information between end-users.

As is apparent from the above, the apparatus and method for predicting the quality of slurry according to one aspect of the present invention can configure a training dataset by linking vibration data measured on the outside of a ball mill facility, temperature data measured in an adhesive manner on the outside of the ball mill facility with the temperature of a slurry measured by inserting the thermometer into a lid of the ball mill facility, and then predict the viscosity, the particle size, and the temperature of the slurry in a ceramic ball mill process based on the adhesive temperature data and the vibration data using AI, thereby minimizing modification of the ball mill facility and enabling easy and convenient prediction of the quality of the slurry on site at a low cost without requiring an expensive measuring device.

Although the present invention has been described with reference to embodiments illustrated in the drawings, the embodiments disclosed above should be construed as being illustrative rather than limiting the present invention, and those skilled in the art should appreciate that various substitutions, modifications, and changes are possible without departing from the scope and spirit of the present invention.

Therefore, the scope of the present invention is defined by the appended claims of the present invention.

What is claimed is:

1. An apparatus for predicting quality of slurry, the apparatus comprising:
a data collection module that collects at least one type of data among raw and secondary material data, process condition data, environmental data, and measurement data in a ball mill process;
a contact temperature sensor attached to an outside of a ball mill facility to measure a facility temperature;
a vibration sensor that detects vibration from the outside of the ball mill facility;
a database; and
a processor operatively coupled to the data collection module, the contact temperature sensor, the vibration sensor, and the database,
wherein the processor stores the data collected through the data collection module together with a contact temperature measured by the contact temperature sensor and vibration data detected by the vibration sensor in the database, then generates and trains a learning model, and then predicts quality of slurry based on the contact temperature and the vibration data in the ball mill process.

2. The apparatus of claim 1, wherein the data collection module includes:
a slurry measuring device that extracts slurry from the ball mill facility to measure a viscometer slurry viscosity, a granulometer slurry particle size, and a viscometer slurry temperature; and
a slurry temperature measuring device that forms a measuring hole in a lid of the ball mill facility and measures a lid hole slurry temperature with a temperature sensor inserted into the measuring hole.

3. The apparatus of claim 2, wherein the measuring hole has a size that allows the slurry to pass therethrough while preventing a ceramic ball from passing therethrough.

4. The apparatus of claim 2, wherein the processor calculates a correction value between the viscometer slurry temperature measured by the slurry measuring device and the lid hole slurry temperature measured by the slurry temperature measuring device, generates a mathematical model through correlation analysis linking the lid hole slurry temperature measured by the slurry temperature measuring device with the contact temperature measured by the contact temperature sensor in a test process, and then predicts a slurry temperature based on the contact temperature of the contact temperature sensor in the ball mill process.

5. The apparatus of claim 4, wherein the processor generates a mathematical model for the lid hole slurry temperature according to a ball mill time, generates a mathematical model for the viscometer slurry temperature according to the ball mill time, calculates an external influence degree through the mathematical model for the lid hole slurry temperature to correct the mathematical model for the viscometer slurry temperature, and calculates a temperature rise of the slurry from the corrected mathematical model for the viscometer slurry temperature to supplement the mathematical model with the lid hole slurry temperature.

6. The apparatus of claim 1, wherein the quality of the slurry includes at least one of a slurry viscosity, a slurry particle size, and a slurry temperature.

7. A method of predicting quality of slurry, the method comprising:
collecting, by a processor, at least one type of data among raw and secondary material data, process condition data, environmental data, and measurement data through a data collection module in a ball mill process;
storing, by the processor, the collected data in the database;
generating and training, by the processor, a learning model based on the data stored in the database;
receiving, by the processor, a contact temperature and vibration data; and
predicting, by the processor, quality of slurry based on the contact temperature and the vibration data through the learning model.

8. The method of claim 7, wherein the predicting of the quality of the slurry includes:
calculating, by the processor, a correction value between a viscometer slurry temperature measured by a slurry measuring device and a lid hole slurry temperature measured by a slurry temperature measuring device;
generating a mathematical model through correlation analysis linking the lid hole slurry temperature measured by the slurry temperature measuring device with the contact temperature measured by a contact temperature sensor in a test process; and
then predicting a slurry temperature based on the contact temperature of the contact temperature sensor in a ball mill process.

9. The method of claim 8, wherein the predicting of the quality of the slurry includes:
generating, by the processor, a mathematical model for the lid hole slurry temperature according to a ball mill time;
generating a mathematical model for the viscometer slurry temperature according to the ball mill time;
calculating an external influence degree through the mathematical model for the lid hole slurry temperature to correct the mathematical model for the viscometer slurry temperature; and
calculating a temperature rise of the slurry from the corrected mathematical model for the viscometer slurry temperature to supplement the mathematical model with the lid hole slurry temperature.

10. The method of claim 7, wherein the quality of the slurry includes at least one of a slurry viscosity, a slurry particle size, and a slurry temperature.
